(19) United States
(12) Patent Application Publication
(10) Pub. No.: US 2018/0049906 A1
(43) Pub. Date: Feb. 22, 2018

(54) A Method for Producing a Brace, the Brace as Such, and a Method to Fix the Position of a Broken Bone in a Limb

(71) Applicant: MBRACE MEDICAL B.V., Tegelen (NL)

(72) Inventor: Heinrich Mark Jan Janzing, Tegelen (NL)

(21) Appl. No.: 15/555,895

(22) PCT Filed: Mar. 7, 2016
(86) PCT No.: PCT/EP2016/054737

§ 371 Date: Sep. 5, 2017

(30) Foreign Application Priority Data
Mar. 8, 2015 (NL) 2014417
Oct. 3, 2015 (NL) 2015555

(51) Int. Cl. A61F 5/058; G06F 19/00

(52) U.S. Cl. CPC A61F 5/05858; A61B 5/1075; G06F 19/3437

(57) ABSTRACT

A method to produce a brace for fixing a position of a broken bone in a first limb, includes the steps of making an image of a residual limb that corresponds to the first limb, and producing the brace by making a three-dimensional appliance that has dimensions that correspond to the image of the residual limb and which is constituted to adjustably enclose the broken limb.

### GENERAL FIELD OF THE INVENTION

The present invention pertains to a method to produce a brace for fixing a position of a broken bone in a limb and to a brace produced with such a method. The invention also pertains to a method for actually fixing a position of a broken bone in a limb.

### BACKGROUND OF THE INVENTION

A broken bone in a limb, such as a broken wrist of an arm, is usually treated in a hospital accident and emergency department. The treatment differs depending on the severity of the injury. As a start of the treatment painkillers may be given and then a doctor or other medical schooled person may fix a splint (i.e. a rigid device used to prevent motion of the ends of the fractured bone) to the limb to secure it in position and prevent further damage. In most cases an X-ray will be taken of the limb to see what kind of fracture it is. Even hairline fractures should show faintly on X-ray.

The type of treatment of the broken bone depends largely on the type of fracture. More complex fractures are often treated with surgery to realign (reduce) and fix the broken bones. This includes displaced fractures, fractures involving a joint, and open fractures. Surgeons can fix bones with wires, plates, screws or rods. This is called open reduction and internal fixation (ORIF). Any metalwork is usually not removed unless it becomes a problem. In rare cases an external frame is used to hold the broken bones, known as an external fixator. After surgery, often a plaster cast is applied to protect the repair. A sling may also be provided for comfort. Surgical methods of treating fractures have their own risks and benefits, but usually surgery is done only if conservative treatment (treatment without surgery) has failed, is very likely to fail, or likely to result in a poor functional outcome. In other words, surgery is only applied in cases where it appears absolutely necessary, and only if the patient is fit for surgery. In other cases, no surgery is applied.

A very simple fracture where the bone remains aligned can be treated simply by applying a plaster cast. This holds the broken ends together so they can heal. With more severe fractures, the bones can become misaligned (displaced). If the bone is not reduced, the bones will not heal well. Therefore in many cases a technique called ‘closed reduction’ to pull the bones back into position is used. Local or regional anesthetic will be used to numb the arm, or the patient (especially children) will be put to sleep using a general anaesthetic. If the bones' new position is found to be OK by the person who performs the reduction, the limb is treated with a plaster cast to fix this position. In the case of broken bones in a limb, typically there is a lot of swelling involved, which swelling may last up to several days or even longer. The swelling is typically more severe when a bone is broken in the lower half of a limb (i.e. the lower arm or lower leg), in particular when a bone is broken in the distal part of that lower limb, even further in particular when a bone in the actual joint (wrist, ankle) or a site adjacent the joint is broken. In the latter cases (which are the most common), the swelling may last for up to two weeks or even longer. Therefore, in case of fixing a broken bone in a limb, the initial brace is often removed after some time and replaced with a brace having a smaller cross-section such that the limb is nicely enclosed again. A patient can go through several brace replacements depending on the type of break. In particular in case of a broken joint or broken bone in the distal half of a limb, two to three replacements to take into account the disappearance of the initial swelling are common. Apart from the time this takes and stress for the patient, the inherent disadvantage is that in a significant time period (typically days) before the brace is actually replaced, there is often no good enclosure of the limb which may lead to dislocation of the broken bone. In order to prevent this, the brace should be replaced more regularly. However, this is not feasibly economically nor from a view of patient stress. US 2010/0138193 (Summit et al.), with reference to FIGS. 32-37, recognises this problem and proposes in section [0115] through [0120] a modular brace of which the sections can be removed sequentially as the patient heals. However, after removal of a non-fitting module the support at that site is lost which may be very disadvantageous. The proposed additional padding to take into account shrinkage of a limb is also not able in practice to compensate for each and every decrease in swelling, while at the same time maintaining sufficient support for the broken bone in the limb.

In practice, typically about 90% of the broken arms are treated without surgery. This does not mean that the type of fracture is not complex in all of these cases. For example elder patients, although the type of fracture would necessitate surgery to obtain a fully functional limb after the broken bone has healed, are often not fit for (bone) surgery. The limb therefore is often treated conservative, since the estimated dis-functioning does not outweigh the risk of surgery. In younger patients on the other hand, the conservative treatment also leads to problems as explained here above, but also for example evolving from the fact that the commonly known plaster cast leads to severe restrictions in using of and caring for the arm whilst being supported by the plaster cast. Also, the proper position of the bones after reduction is often (partly) lost while applying a plaster cast, which may lead to partial loss of functionality of the arm after the broken bone has healed.

### OBJECT OF THE INVENTION

It is an object of the invention to provide a method to produce a brace for fixing a position of a broken bone in a limb, which brace can be used in conservative treatment of the limb, and which does not, or at least to a lesser extent, suffer from the disadvantages of prior art treatment methods. It is another object of the invention to provide for a method for fixing a position of a broken bone in a limb that overcomes or mitigates the disadvantages of any prior art methods correspondingly.

### SUMMARY OF THE INVENTION

In order to meet the first object of the invention, a method as outlined in the GENERAL FIELD OF THE INVENTION section here above has been devised, the method comprising -making an image of a residual limb that corresponds to the first limb, and producing the brace by making a three-dimensional appliance that has dimensions that correspond to the image of the residual limb, and which is constituted to adjustably enclose the broken limb.

It was applicant's recognition that a (near) perfect, personalized brace for the limb with the broken bone can be produced by using the residual, but healthy (unbroken) limb as a model. The residual limb (when having intact bones) has a three-dimensional shape that corresponds to the shape the broken limb should ultimately get after (near) perfect, error free reduction. By imaging the residual limb and producing a brace based on this image, the (near) ideal brace for the limb with the broken bone may be obtained. Advantageously, the swelling of the limb with the broken bone is taken into account. Waiting with applying the brace until the swelling is gone is no option in case of a limb with a broken bone that needs to be fixed, in particular since significant swelling may last days or even more than a week. To this end, in a first embodiment of the present invention the constitution of the brace is such that it adjustably encloses the broken limb. By providing the brace with an adjustable enclosure (by having an adjustable cross-sectional shape), a (medically trained) person is able to control the enclosure by using external forces to reach a predetermined three-dimensional shape wherein the limb is nicely enclosed independent of the level of swelling. In other words, the form and/or spatial arrangement of the part or parts of which the brace is constituted can be adjusted (by a human person), such that the broken limb is continuously enclosed, no matter what the amount of swelling is. Independent of the cross sectional shape of the broken limb at the time of reduction and during healing, it is possible to adjust the brace intermittently to provide for the continuous enclosure of the limb. The compliance of the soft tissue, including the swollen tissue, will suffice to make sure there is a continuous enclosure even though the adjustments are made only intermittently. In practice, the adjustments will most likely be performed for example every 2 or 3 days, but in any case, they can be done at points in time far more frequently when compared with the typical time periods in between complete renewals of the brace (2 weeks). Depending on the frequency, an optional thin padding may be enough to adjust to the changes in shape of the limb (mainly a reduced cross section due to a decreased swelling) even at a low frequency of adjustment, such that in practice there is a true continuous enclosure of the limb, and hence an ideal support and fixture of the reduced bone, even when the adjustments are made for example less frequent than every 2-3 days.

In case of an image that has more than one dimension, a brace that corresponds to the mirror image of the image made of the residual limb can be used.

The invention is also embodied in a method to produce a brace for fixing a position of a broken bone in a first limb, comprising making a three-dimensional image of a residual limb that corresponds to the first limb, and producing the brace by making a three-dimensional appliance having dimensions that correspond to a mirror image of the said three-dimensional image. Although the adjustable enclosure as described here above is advantageous, it was surprisingly found that even without this feature the brace has its medical advantages. In particular in those cases were swellings are not extremely high, or for example very even across the limb, a brace that is a simple mirror image of the residual limb can be used to fix the position of the broken bone.

The invention is also embodied in a brace produced with the new method. In particular, the invention is embodied in a brace for fixing a position of a broken bone in a first limb, the brace having a shape that corresponds to the shape of a residual limb that corresponds to the first limb, the brace comprising two separate support elements that have an adjustable mutual spatial configuration to provide for a constitution to adjustably enclose the broken limb.

The invention is also embodied in a method for fixing a position of a broken bone in a first limb comprising producing a brace as explained here above, whereafter the brace is placed around the first limb independent of an amount of swelling of the limb. Preferably the brace is place around the limb within 24 hours after the bone in the limb was broken, preferably even within 6, 5, 4, 3, 2 or 1 hour after that occasion.

Each of the specific embodiments as described below in the section titled “EMBODIMENTS”, can also be used in conjunction with each of the above described basic embodiments according to the invention.

### DEFINITIONS

A brace for fixing a position of a broken bone is a rigid orthopedic appliance used to support a bodily part comprising the broken bone in a fixed position.

To enclose an object means to surround the object while being contiguous with the surface of this object in order to fit this object.

An image of an object is a representation of the external form of that object. An image is often two-dimensional, but may for example also be one or three-dimensional.

To adjustably enclose an object means that the enclosure of which the cross-sectional shape can be controlled by using external forces to reach a predetermined three-dimensional shape wherein the object is enclosed. This is opposed to for example using an elastic brace (or other deformable material) to provide for an enclosure of an object, in which case the brace simply takes the shape of the object as it is (no external forces needed), instead of taking a predetermined shape that can be adjusted by using external forces, and opposed to for example a modular brace have a fixed cross-sectional shape, wherein modules can be removed to change the length of the circumference that is contiguous with the limb, but wherein the cross-sectional shape imposed by the remaining modules remains the same.

### EMBODIMENTS

In a first embodiment of the invention the appliance comprises two separate support elements that have an adjustable mutual spatial configuration to provide for the adjustable enclosure of the limb. In this embodiment, as opposed to a traditional one piece circumferential brace, the brace comprises two separate support elements to enclose the limb and therewith fix the position of the broken bone. The advantage is that it is easier to apply the brace, and easier to fine tune the enclosure to perfectly fit the limb. Another advantage is that the brace as a whole may be less bulky, easier to wear and easier to use in everyday practice. In a further embodiment the two separate support elements are configured to support the broken limb at opposing sides thereof, which provides for an adequate and comfortable enclosure of the limb. In another further embodiment one of the support elements has two separate supporting surfaces to be contiguous with a surface of the first limb. By constituting at least one of the support elements out of two separate sub-elements, there is even more flexibility to provide for a perfect enclosure of the limb.

In another embodiment of the present invention the image is a three dimensional image. Although it may be feasible to use a one dimensional image (for example the circumferential length of the wrist of the residual arm, which is found to be representative for the shape of the lower arm), or a two dimensional image (for example by combining the circumferential length of the wrist of the residual arm with a dimension of the lower arm perpendicular to the length of the arm), it was found that by making a three-dimensional scan of the residual limb and producing a brace for this limb, a near perfect brace can be designed for the limb having the broken bone. In this case, the brace has dimensions that correspond to the (lateral) mirror image of the residual limb. The difference with an actual 100% perfect brace results only from the minor differences in the mirror image of the residual limb with the first limb before the bone thereof was broken. Such differences are negligibly small for the vast majority of people. This means that this embodiment of the present invention can be used for almost all patients. When using the newly created brace for the limb with the broken bone, the limb can be supported in the best three dimensional way while having regard of the position of the broken bone. Of course, it may still be needed to reduce the broken bone to its best possible position, but once reduced, the brace provides for an enclosure that is perfect for keeping the broken bone in this position. As the skilled person would understand, in this embodiment it is not essential at which stage a mirror view of the residual limb is created. In an embodiment for example, a 3D model of a brace is formed that perfectly fits the residual limb, and then this model is mirrored into the model to produce the actual brace needed. In another embodiment the three dimensional image of the residual limb itself is mirrored, whereafter a brace is modeled using this mirrored image of the residual limb. In both instances the ultimate brace has dimensions that correspond to a mirror image of the said three-dimensional image of the healthy residual limb.

In another embodiment of the method according to the invention, before the three-dimensional image of the residual limb is made, this residual limb is brought in a predetermined position, after which the image is made while the residual limb takes the said predetermined position. By bringing the residual limb in a predetermined position, an ideal model can be provided for the ultimate brace. For example, when the limb is an arm, the predetermined position could be a completely stretched arm, instead of an arm wherein the wrist angles with respect to the forearm. In a preferred embodiment the first limb is brought in a position that corresponds to the said predetermined position, before the brace is placed to support the first limb. This way, it is made sure that the brace is optimally dimensioned for the limb with the broken bone. Further preferred is a method wherein the predetermined position is obtained by applying a traction force to the corresponding limb. For example, a residual arm and first (broken) arm could be subjected to a commonly known finger trap or Weinberger hand traction.

In yet another embodiment wherein the brace has a common longitudinal shape and a substantial circular circumference, the brace has a longitudinal cleavage. Normal plaster brace regularly have no cleavage since they are cast as a splint or are cast around the limb in situ. However, a cleavage could be advantageous for several reasons, in particular to provide the opportunity for deformation at a site of swelling. In the novel method wherein a brace is formed without depending on the broken limb as a model, a cleavage could easily be incorporated in the brace. The cleavage could also help in making it easier to place the brace around the broken limb. In particular, when in an embodiment wherein the cleavage runs along the entire length of the brace, the brace can actually be opened (slightly) so that it can be easily guided to slide over the limb. An important advantage of a cleavage that crosses the entire length of the brace is that the brace can be adapted to circumstances that may occur during the wearing of the brace such as swelling of the limb, the need for inspection of the arm, the need for treating the skin etc. In a further embodiment of a brace with a cleavage, the brace is provided with a closure means to close the circumference of the brace at the cleavage. This way it can be prevented that the cleavage leads to the brace not, or no longer, grasping the limb with sufficient pressure. In a preferred embodiment the closure means and brace are produced as a unit. For example, both sides of the cleavage of the brace could be provided with appliances that mate to form a durable closure that may even allow unlocking (for example two items that can be simply clicked together, or form a luer lock type closure etc.).

It is foreseen that a brace is made from an elastic material. For example, if local swellings occur, an elastic material may deform locally to provide that the pressure on the tissue becomes very high. Also, an elastic material provides for an increased level of comfort for the person who wears the brace. Indeed, the elastic modulus of the material of the brace (i.e. the resistance of the brace to being deformed elastically) should have a value such that the brace on the one hand is rigid enough to provide for a durable fixation of the broken bone, but on the other hand is capable to allow local deformation as a result of common tissue swelling. It is an option to produce the brace from a material that has a relatively low elastic modulus where it is in contact with the patients skin, and a high elastic modulus on the outer circumference to provide for the required rigidity. In an alternative embodiment the brace is produced as a multi layered structure, each layer having tuned physical properties (e.g. a very compliant inner layer and a very rigid outer layer).

In still another embodiment the first limb is an arm having a distal radius fracture. It is believed that the present invention is ideally suitable for this kind of fracture. This is based on the fact that the arms commonly have the required similarity in 3D shape, and also, given the fact that the bones in the arm break relatively easily (when compared to the bones in a leg) and in many cases are treated conservatively with a plaster cast, resulting a partial loss of normal functionality.

In again another embodiment the three-dimensional image is created using a stereoscopic scanner, laser scanner or photogrammetric scanner. Although a simple 2D scanner could be used to make a 3D image, for example by taking multiple images from multiple angles, one of the mentioned 3D scanners is preferred given the ease of use and reliability of the outcome.

In still again another embodiment the brace is made using a 3D printer, for example using Selective Laser Sintering (SLS) or Fused Deposition Modelling. 3D printing is a simple technique that has evolved to become a technique for rapid producing unique appliances of sufficient strength. It gives the designer many ways of freedom to produce the ultimate product, and also, to use materials that are elastic when hardened, resistant to moist, heat and mechanical load.

The material for producing the brace should be chosen such that the brace does not generally invoke skin necrosis, a rash or any allergic reaction. Preferably the material is odourless, and also, the printing process itself does not generate any odours or toxic substances. This way, the scanning, control and printing functions can be done in one room in a hospital.

Preferably the material for producing the brace is a (curable) polymer or other substance that is easy to recycle or even biodegradable. Typical polymers envisioned for this purpose are (co)polyurethane(s), (co)polycarbonate(s), (co)polyester(s), (co)polyamide(s), (co)poly(ester-amide(s), mixtures thereof and/or copolymers thereof. Biodegradable polymers are polymers that will decompose in natural aerobic (e.g. composting) and/or anaerobic (e.g. landfill) environments. They may be composed of either biopolymers, which may be naturally produced polymers or polymers whose components are derived from renewable raw materials, but may also be petroleum-based, or a blend of one or more of these types of polymers. Most aliphatic polyesters are biodegradable due to their potentially hydrolysable ester bonds. Typical examples of naturally produced biodegradable polymers are polyhydroxyalkanoates (PHA's) like poly-3-hydroxybutyrate (PHB), polyhydroxyvalerate (PHV) and polyhydroxyhexanoate (PHH), and starch, cellulose, keratin and derivatives thereof. A biodegradable polymer from a renewable resource is for example polylactic acid (PLA). Examples of other synthetic biodegradable polymers are polybutylene succinate (PBS), polycaprolactone (PCL), polyvinylacetate (PVA) and cellulose esters like cellulose acetate, nitrocellulose and their derivates such as celluloid.

The invention will now be further explained using the following figures and examples showing specific embodiments of the invention.

### EXAMPLES

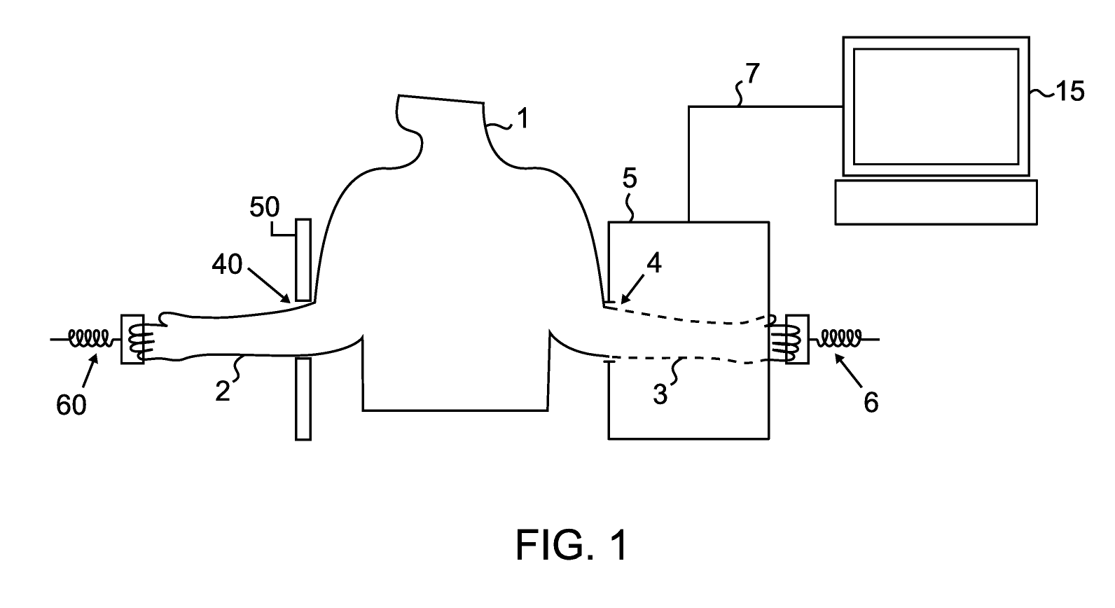

FIG. 1 provides a schematic view of a patient having a broken arm undergoing a method according to the present invention.

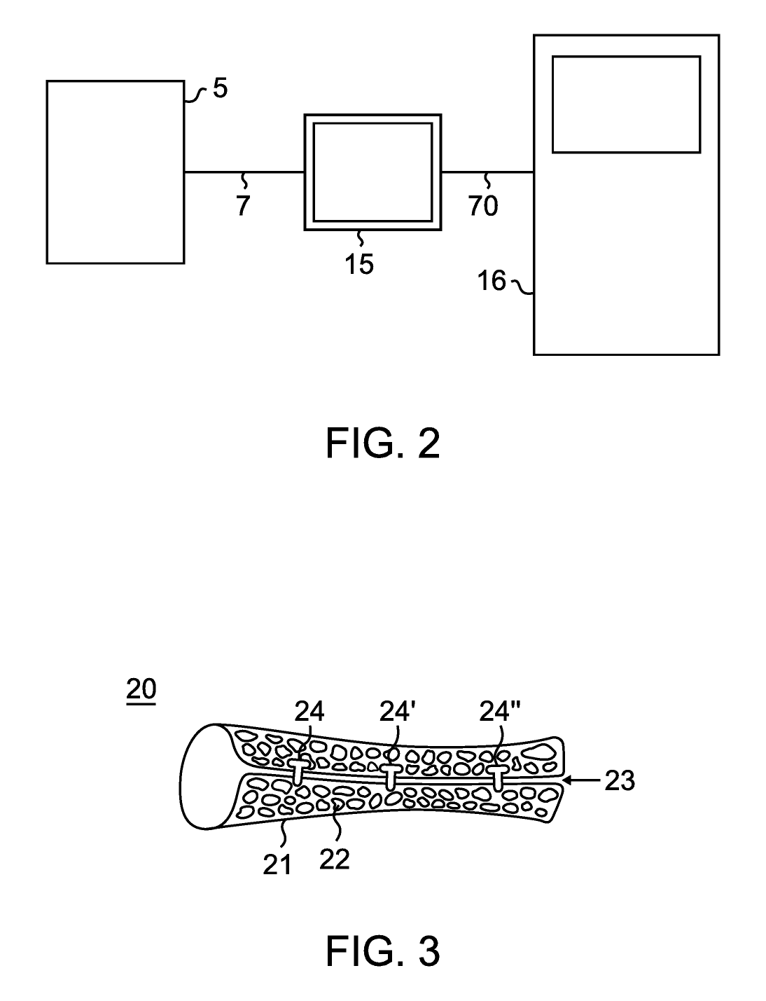

FIG. 2 provides a schematic representation for a system to perform the method according to the invention.

FIG. 3 provides a schematic representation of a brace produced according to the invention.

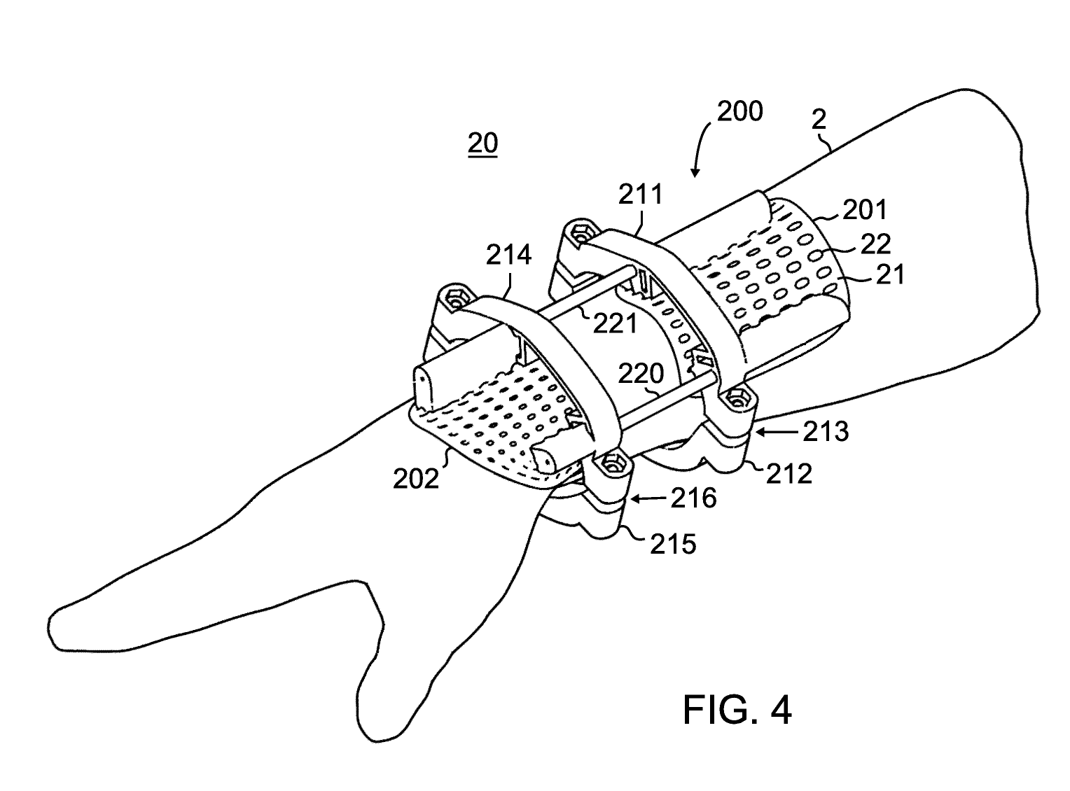

FIG. 4 provides a schematic representation of another brace produced according to the invention.

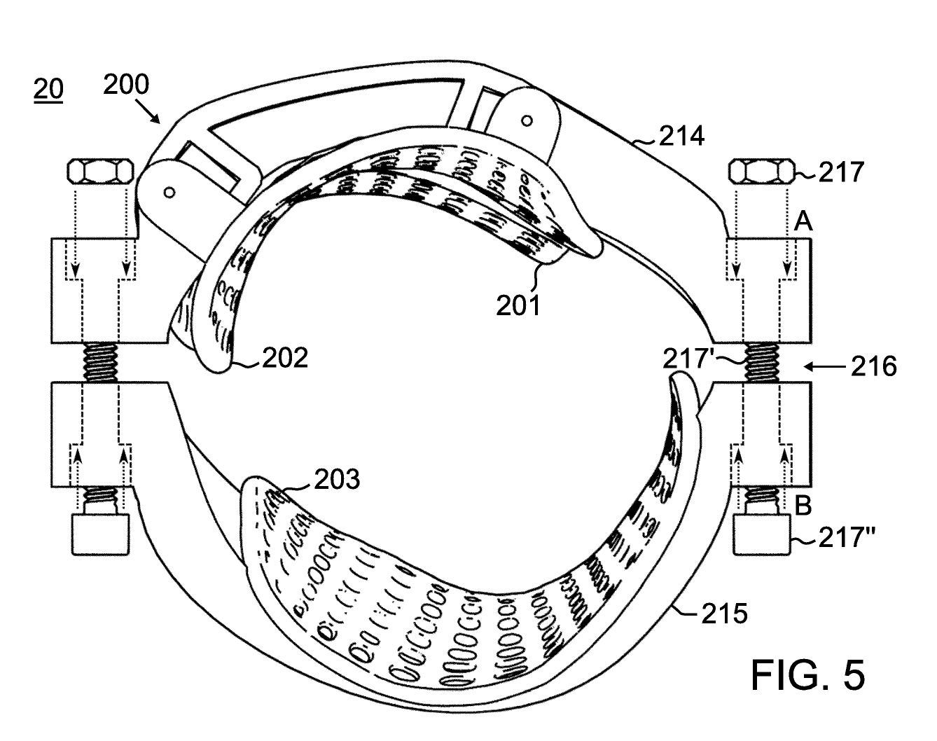

FIG. 5 provides a front view of the brace of FIG. 4.

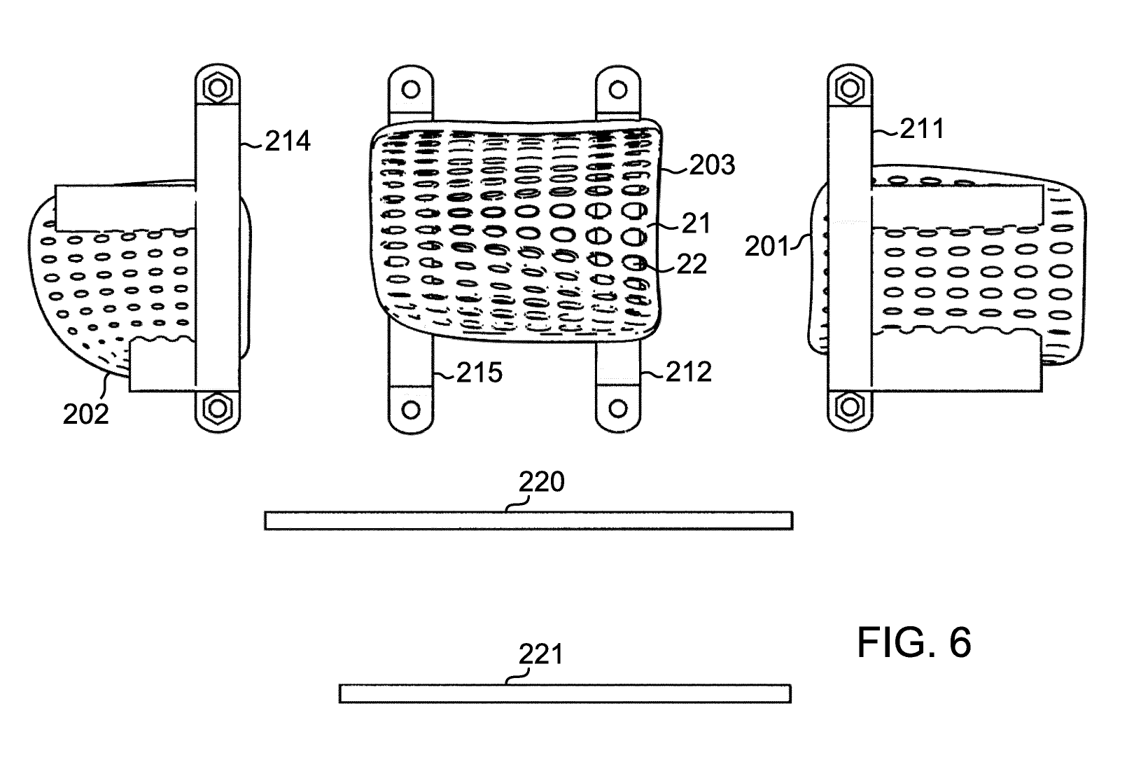

FIG. 6 shows the basic parts of the brace of FIG. 4.

Example 1 assessment of symmetrical shape of corresponding limbs

Example 2 describes a test with live subject having healthy limbs to assess the fitting of a brace according to the invention.

Example 3 describes a human cadaver study with a brace according to FIG. 4.

FIG. 1

FIG. 1 provides a schematic view of a patient 1 having a broken arm 2, in this case a distal radius fracture needing (closed) reduction and fixation by an external brace for proper healing. Firstly, the patient 1 puts his residual (unbroken) arm 3 in a stereoscopic scanner 5. The opening 4 of this scanner, in combination with the finger trap traction device 6 make sure that the forearm is brought in a predetermined position. Alternatively, the arm is broken in a scanner while resting on a standard support to make sure the arm obtains a predetermined positon (for such an alternative process a standard scanner such as the laseroptic Footin3D scanner available from Elinvision, Biruliskiu village, Lithuania) can be advantageously used.

The process is monitored using central computer 15 (having a wireless connection 7 with the scanner) which is operator controlled. As soon as the computer establishes, by analysing pre scans of scanner 5, that the forearm is in the proper position, the patient is told by the operator to keep still and a three dimensional scan of the forearm of arm 3 is made. The resulting three-dimensional image of the forearm is processed in the computer 15 and mirror imaged. This way the three dimensional image of the residual arm 3 is turned into an (imaginary) three dimensional image of the broken arm 2. In a next step, a computer model of a brace is (automatically) designed. This brace has dimensions that correspond to the image of the residual limb since its dimensions fit the dimensions of the mirror image of the residual arm. This design of the brace is sent to a 3D printer (see FIG. 2), such as for example a Fortus 250mc as available from Stratasys, Eden Prairie, Minn., United States. This printer is able to rapidly print any appliance from a material having the required rigidity and elasticity for a brace after hardening. By using the mirror image of the residual limb as a model for the appliance, it is provided that the newly designed three-dimensional appliance has (inner) dimensions that exactly correspond to a mirror image of the said three-dimensional image, i.e. this appliance neatly fits an imaginary arm having the spatial configuration of this mirror image. Since the normal 3D form of the broken arm correspond to the mirror image of the residual arm (see Example 1 here below), the printed appliance inherently is a brace that corresponds to the arm 2 after proper reduction. The whole process of scanning and printing, depending mainly on the required size of the brace and the type of ink, may take between 10 and 60 minutes using a common 3D printing apparatus such as the ones used for rapid prototyping.

After the brace (see FIGS. 3 and 4) has been produced, the computer provides instructions to guide the patient and medic to bring the broken arm 2 in a position that corresponds to the position the arm 3 took in the scanner 5. For this, the patient puts his arm through opening 40 of wall 50, where after a finger trap traction device 60 is applied. The opening 40, wall 50 and finger trap traction device 60 correspond to the opening 4 of scanner 5 and finger trap traction device 6. This way the reduced arm 2 can be brought in a position that corresponds to the position arm 3 had while being scanned. Alternatively, the broken bone is slightly over corrected to ensure that it sets into the exact right position after the brace has been placed around the arm. Another possibility is to design a brace that has such an over correction incorporated, or to apply both types of overcorrection. In any case, in a final step the brace is placed around arm 2 to support this arm and fix the position of the broken bone.

FIG. 2

FIG. 2 provides a schematic representation for a system to perform the method according to the invention. In this view, scanner 5 is depicted while being in operative connection via wireless line 7 with computer 15. This computer 15 on its turn is in operative connection via a wireless line 70 with 3D printer 16.

FIG. 3

FIG. 3 provides a schematic representation of a brace produced according to the invention. In this case the brace 20 is a longitudinal brace of the type used for supporting a forearm. The brace is made from an ink that after curing (hardening) is elastic, but which has an elastic modulus of a value such that the brace is capable to provide sufficient overall rigidity to provide for spatial fixation of the reduced bone. The brace is provided with a cleavage 23 over its entire length. In this case the cleavage runs almost parallel to the longitudinal axis of the brace. In order to be able and close the brace, closure means 24, 24′ and 24″ are provided which means are formed integrally with the brace by the 3D printer. The closure means are adjustable such that the brace when being closed can leave an opening at the site of the cleavage or can be completely closed. This way the brace can adjustably enclose the broken limb. By having the brace made as a constitution with an adjustable enclosure, the amount of swelling can be taken into account when fitting the brace around the broken limb and still have an adequate enclosure. Right after the bone is broken, the amount of swelling is typically high and the brace may be positioned with the cleavage wide open. With the swelling going away, the cleavage may be gradually closed to arrive at a corresponding continuous enclosure of the limb.

The material of the brace has no sharp edges, is provided in a fish-net like structure of rigid ribbons 21, leaving openings 22. This makes the brace very light when compared to common plaster casts. Also, the cured material (which is typically a polymer) can be chosen to be water resistant, such that bathing with the brace is an option. The openings 22 make sure the arm can dry after bathing. It is also foreseen that the brace is provided with a separate element, such as a metal or carbon fibre reinforced rod, to provide for additional rigidity if needed. This provides for more freedom in choice of printing ink (or, in case 3D printing is not the method of choice, more freedom in choosing this method and the material used). Such an element can be incorporated in the 3D print or attached after printing.

In an alternative embodiment closure means are used that are separate from the brace itself such as for example ty-wrap like ribbons. This provides that the brace is easier (and faster) to produce. Also, the closure means can be chosen such that they cannot be opened by the patient himself.

FIG. 4

FIG. 4 provides a schematic representation of another brace produced according to the invention, which is a brace that is easier to adjustably enclose different cross sectional shapes of the broken arm 2 when compared with the brace of FIG. 3. The brace 20 in this case comprises two separate support elements that have an adjustable mutual spatial configuration to provide for the adjustable enclosure of the arm. The first of these support elements, element 200 comprises two separate supporting surfaces 201 and 202 which are positioned on top of the broken arm 2 to be contiguous with its upper surface. Each of these supporting surfaces has a structure of rigid ribbons 21 that surround holes 22 (cf. structure of brace of FIG. 3). These supporting surfaces are interconnected via rods 220 and 221 that are slidably connected into clamping ribs 211 and 214 respectively. On the opposing underside the second support element 203 is present (not denoted in FIG. 4; see FIGS. 5 and 6). Of this support element 203 only the clamping ribs 212 and 215 can be seen in the bird's eye view of FIG. 4. Both elements (the element that is comprised of support surfaces 201 and 202, and the element 203) together are configured to support the broken arm at opposing sides thereof.

The brace 20 is a three-dimensional appliance that has dimensions that correspond to the image of the residual arm (not shown in FIG. 4) and the constitution is such that the arm is adjustably enclosed. For this, opposing clamping ribs can be moved towards and away from each other, decreasing or increasing open space 213 and 216 respectively. For this use is made of screw means 217 (not shown in FIG. 4; see FIG. 5). By increasing or decreasing the space using screw means 217 the enclosure of the arm is controlled. The external forces applied to the screw means can be used to reach a predetermined three-dimensional shape for the brace, so that the arm is nicely enclosed independent of the level of swelling of the arm. Local (typically very small) swelling can be taken into account by using a padding on the supporting surfaces.

FIG. 5

FIG. 5 provides a front end view of the brace of FIG. 4. In this front end view, lower support element 203 can be seen with its clamping rib 215 attached to the support surface. The upper element 200 with its support surfaces 201 and 203 is also depicted, including clamping rib 214 that opposes clamping rib 215. As can be seen in FIG. 5, screw means 217 (217′, 217″) are present to adjust the space 216 between the two clamping elements (indicated with arrows A and B). This way the cross-sectional dimension of the brace can be adjusted to makes sure the arm is enclosed independent of the level of swelling.

FIG. 6

FIG. 6 shows the basic parts of the brace of FIG. 4 while of being assembled to form the brace 20. Depicted are support element 203 with its clamping ribs 212 and 215, as well as the supporting surfaces 201 and 202 that together form the second support element 200 using rods 220 and 221 to connect the two support surfaces.

### Example 1

This example describes an assessment of symmetrical shape of corresponding limbs. For this assessment the dimensions of the lower arm of 56 healthy adults who did not previously had a fracture in their lower arm was measured by one medically trained practitioner. Of the subjects the following measures were taken: 1) the circumferential length of the hand at the first webspace, 2) the smallest circumferential length around the wrist adjacent (distally) of the ulnar styloid, 3) the smallest circumferential length of the lower arm, proximal of the wrist, 4) the circumferential length of the lower arm at the elbow crease and 5) the distance between the lateral epicondyle to the styloid radius process. The results indicated that although measures 1) through 4) are on average slightly higher for rights arms then for left arms, the overall resemblance of the measures is very high. This shows that an image of one arm can be sued to create a brace for the other arm.

### Example 2

Example 2 describes a test with live human subjects having healthy limbs to assess the fitting of a brace according to the invention. For this test a first subject, healthy male, 54 years of age, was used. Of this subject one arm was scanned to deliver a three-dimensional image of that arm. Based on that image, a brace was made according to FIG. 4. This brace was provided with Delta-Dry softliner (available from BSN Medical Inc., Charlotte, N.C., USA) and worn by the subject for 4 days. Apart from some very light red spots, no clinical signs could be observed. The brace was comfortable to wear and after showering dried very quickly.

As a comparison, a second subject, a healthy female, 56 years of age and a third subject, a healthy male of 31 years of age, wore corresponding braces (see FIG. 4), but not modelled using an image of one of their own arms, but using a normalised model of an arm. It appeared that the brace was not able to provide optimal support against rotation of the arm and also, even after a mere 24 hours induced local clinical signs on the lower arm (red skin and indentations) in particular radial dorsal and ulnar volar.

### Example 3

Example 3 describes a human cadaver study with a brace according to FIG. 4. For the study multiple cadaver arms of two types are used, fresh frozen and models created using Anubifix® balsam (Erasmus University, Rotterdam, The Netherlands). Each arm is imaged to create a corresponding brace. Before the bone in each of the arms is broken, each arm is tested for its mobility in the wrists by performing a standardised test on the arm. The results are recorded by pictures in a situation of maximum flexing and maximum extension of the wrist. Thereafter standardised X-ray images are taken of the wrist in two directions. This way each arm is imaged with the bone intact. Then a Colles' fracture is made with osteotomic surgery via dorsal entry of the arm (as described by Baumbach et al. in BMC Musculoskeletal disorders, 2012, 12-252; “Assessment of a novel biomechanical fracture model for distal radius fractures”). The arm is closed after the surgery. Then the arm is subjected to the same test as described before and the same pictures and X-ray images are made. This should show the Colles' fracture. If so, the brace is mounted (if needed after reduction of the broken bone), while the arm is under manually applied tensile stress, to enclose the arm. Thereafter, the arm is again subjected to the same test, and pictures and X-rays are being made. If needed, the enclosure the brace is adjusted to fit the arm and the test is repeated. If the broken bone keeps its position, the arm is brought in maximum flexion and maximum extension for twenty times in a row and the test is again repeated to check the fixation of the bone The results indicate that an adequate fixation of the position of a broken bone in an arm can be obtained.

The brace according to the current invention in its broadest sense, can be constituted as a relatively open structure which has several advantages. The first type of advantages are practical ones, less enclosure inherently means a lighter brace, easier to wear and less risk of irritation and rashes. Secondly, a relatively open structure provides the possibility to inspect the enclosed limb with the naked human eye, or to make images of the enclosed limb itself, without needing to remove the brace or to use X-ray. In an embodiment a patient could make various images of the enclosed limb, the images for example being taken from multiple angles with a smart-phone, which images can be send to the medical practitioner and after image analysis provide a good basis for deciding whether or not the bones are properly fixed. This could be used to prevent that X-ray images have to be made in each and all cases. To make visual inspection even easier, it is proposed to manufacture the brace of a translucent material. Translucent plastic, as well as translucent materials suitable to act as padding, as such are known in the art and could, when combined, advantageously be used to produce a brace according to the invention, the brace allowing visual inspection through the brace itself.

What is claimed is:

1. A method to produce a brace for fixing a position of a broken bone in a first limb, comprising the steps of:
making an image of a residual limb that corresponds to the first limb, and
producing the brace by making a three-dimensional appliance that has dimensions that correspond to the image of the residual limb and which is constructed to adjustably enclose the broken limb.

2. A method according to claim 1, wherein the step of producing includes the step of creating the appliance from two separate support elements that have an adjustable mutual spatial configuration to provide for the adjustable enclosure of the limb.

3. A method according to claim 2, wherein the step of producing includes the step of configuring the two separate support elements to support the broken limb at opposing sides thereof.

4. A method according to claim 2, wherein the step of producing includes the step of forming one of the support elements to have two separate supporting surfaces to be contiguous with a surface of the first limb.

5. A method according to claim 1, wherein the step of making includes the step of making the image as a three dimensional image.

6. A method according to claim 1, further comprising the step of, before the image is made, bringing the residual limb in a predetermined position, after which the image is made while the residual limb takes said predetermined position.

7. A method according to claim 6, further comprising the step of bringing the first limb in a position that corresponds to said predetermined position, before the brace is placed to support the first limb.

8. A method according to claim 6, wherein the step of bringing includes the step of obtaining the predetermined position by applying a traction force to the corresponding limb.

9. A method according to claim 1, wherein the first limb arm has a distal radius fracture.

10. A method according to claim 1, wherein the step of making includes the step of making the image as a three-dimensional image by one of the following:
a stereoscopic scanner,
a laser scanner and
a photogrammetric scanner.

11. A method according to claim 1, wherein the step of producing includes the step of producing the brace using a 3D printer.

12. A method to produce a brace for fixing a position of a broken bone in a first limb, comprising the steps of:
making a three-dimensional image of a residual limb that corresponds to the first limb, and
producing the brace by making a three-dimensional appliance having dimensions that correspond to a mirror image of said three-dimensional image.

13. A brace produced with a method according to claim 1.

14. A brace for fixing a position of a broken bone in a first limb, the brace having a shape that corresponds to a shape of a residual limb that corresponds to the first limb, the brace comprising two separate support elements that have an adjustable mutual spatial configuration to provide for a construction to adjustably enclose the broken limb.

15. A method for fixing a position of a broken bone in a first limb, comprising the steps of providing a brace according to claim 13, and placing the brace around the first limb independent of an amount of swelling of the limb.

16. A method according to claim 15, wherein the step of placing including the step of placing the brace within 24 hours after the bone in the limb was broken.

17. A method for fixing a position of a broken bone in a first limb, comprising the steps of providing a brace according to claim 14, and placing the brace around the first limb independent of an amount of swelling of the limb.

18. A method according to claim 15, wherein the step of placing including the step of placing the brace within 24 hours after the bone in the limb was broken.
